(19) United States
(12) Patent Application Publication
(10) Pub. No.: US 2026/0315021 A1
(43) Pub. Date: Oct. 15, 2026

(54) DIGITAL THREE-DIMENSIONAL MEASUREMENT AND EVALUATION METHOD FOR TEMPOROMANDIBULAR JOINTS

(71) Applicant: XuanWu Hospital Capital Medical University, Beijing (CN)

(72) Inventors: Ying ZHAO, Beijing (CN); Zeliang HAO, Beijing (CN); Huanhuan SUN, Beijing (CN)

(21) Appl. No.: 18/996,201

(22) PCT Filed: Aug. 1, 2023
(86) PCT No.: PCT/CN2023/110626

§ 371 Date: Jan. 17, 2025

(30) Foreign Application Priority Data
Aug. 4, 2022 (CN) 202210930059.5

(51) Int. Cl. A61B 6/51; A61B 6/00; A61B 6/03; A61B 6/40; A61B 6/46; A61B 6/50

(52) U.S. Cl. CPC A61B 6/51; A61B 6/032; A61B 6/4085; A61B 6/461; A61B 6/505; A61B 6/5217

(57) ABSTRACT

A digital three-dimensional measurement and evaluation method for temporomandibular joints, comprising: (1) establishing a standardized measurement and evaluation procedure: acquiring cone beam (CT) image data of a patient; performing head position correction to obtain three-dimensional image data of a standard head position; generating a plurality of temporomandibular joint two-dimensional measurement films by using the three-dimensional image data of the standard head position and a specific method; respectively measuring and evaluating joints and surrounding structures and position relationships thereof by using the two-dimensional measurement films; and generating a measurement report and a display image to provide data for clinical diagnosis and treatment; (2) performing a standard measurement method: completing the measurement of line distance and angle indexes by determining mark points and reference lines; and (3) performing a standard measurement report and customized display of the image. According to the present solution, the three-dimensional measurement and evaluation procedure is standard and has good reliability; the two-dimensional measurement film generation method is scientific and has good repeatability; the measurement items are complete and rich, such that the representativeness and the longitudinal comparability are good; digital measurement achieves an accurate result; and the measurement report and the image display are visual and clear.

### CROSS-REFERENCE TO RELATED APPLICATIONS

The present application claims priority to the Chinese patent application No. 2022109300595 filed on Aug. 4, 2022 to the China Patent Office, and entitled “DIGITAL THREE-DIMENSIONAL MEASUREMENT AND EVALUATION METHOD FOR TEMPOROMANDIBULAR JOINTS”, the entire content of which is incorporated herein by reference.

### TECHNICAL FIELD

The present application relates to the field of oral healthcare, and more specifically, to a digital three-dimensional measurement and evaluation method for temporomandibular joints.

### BACKGROUND

Temporomandibular joints, as the only joint structure in a cranio-maxillofacial structure, play an important role in oral mastication, pronunciation and other functions. Temporomandibular joint diseases are common oral diseases with a higher incidence rate in populations. In addition to clinical symptoms and signs, the diagnosis of the temporomandibular joint diseases often relies on imaging evidence. Traditional imaging diagnosis for the temporomandibular joints is mostly qualitative. For the measurement research of the temporomandibular joints and surrounding anatomical structures, manual measurement is often used for evaluation, which is time-consuming and laborious, and there is no unified measurement index and normal value range. The repeatability of the evaluation of the morphology, structure, and position relationship of the temporomandibular joints between patients, before and after treatment, and in long-term follow-up is poor, and comparability is lacking.

Digital cephalometric technology refers to the clinical use of three-dimensional cone beam CT (CBCT) to capture cranio-maxillofacial images, followed by three-dimensional reconstruction for digital cephalometry (including three-dimensional calibration, point fixing, line distance measurement, and angle measurement), and is an effective quantitative evaluation method for evaluating soft and hard tissue structures of a cranio-maxillofacial region. However, there is currently no three-dimensional systematic digital measurement and evaluation method and system for the temporomandibular joints and surrounding structures thereof. Due to the inconsistency between image data of a patient's natural head position obtained by conventional three-dimensional cone beam CT and a standard head position, the heterogeneity of temporomandibular joint imaging is high. If the head position of the same patient cannot be guaranteed to be consistent before and after treatment, it is difficult to guarantee the consistency of each measurement plane. Measurement results lack comparability in both horizontal and vertical directions, and accurate evaluation conclusions cannot be drawn. Therefore, head position correction through three-dimensional registration and the generation of standard two-dimensional measurement films are very important in this process.

In addition, there are currently no standard normal values for measuring the structure and symmetry of healthy temporomandibular joints without temporomandibular joint diseases in a large sample population. There are three reasons: (1) there is significant anatomical variation and measurement heterogeneity in temporomandibular joint structures among different individuals, which requires classified measurement and statistics. (2) There is currently no recognized measurement index for evaluating a three-dimensional structure of the temporomandibular joints based on three-dimensional cone beam CT data. (3) There is no complete and systematic measurement tool to measure it accurately and with good repeatability.

For example, Chinese patent application publication CN107080554A discloses a method for measuring temporomandibular joints, in which an oral model of a patient is obtained; and a target image of the patient is determined through the oral model, and upper and lower jaws are separated according to the target image to make the lower jaw free. This solution determines a temporomandibular joint image by guiding the free lower jaw to a corresponding jaw position through matching and overlapping of coronal images in different jaw position states with corresponding coronal images.

However, the technical solution disclosed in the above patent application is to perform measurement through matching of the coronal images, and does not perform direct and accurate measurement.

Therefore, based on three-dimensional cone beam CT image data, establishing a precise, reproducible, and longitudinally-comparable three-dimensional digital measurement and evaluation system and method for temporomandibular joints and surrounding anatomical structures and position relationships thereof will provide more effective tools for scientific research and clinical diagnosis.

### SUMMARY OF THE INVENTION

The technical problem to be solved by the present application is to provide, for the above defects existing in the prior art, a digital three-dimensional measurement and evaluation method for temporomandibular joints which is high in measurement precision, and good in repeatability and longitudinal comparability.

According to the present application, a digital three-dimensional measurement and evaluation method for temporomandibular joints is provided, including:

### 1) Establishing a Standardized Measurement and Evaluation Procedure

first step: acquiring three-dimensional cone beam CT image data of a patient;
second step: performing head position correction on the basis of the three-dimensional cone beam CT image data to obtain three-dimensional image data of a standard head position;
third step: generating a plurality of temporomandibular joint two-dimensional measurement films by using the three-dimensional image data of the standard head position and a specific method;
fourth step: measuring and evaluating temporomandibular joints on both sides and surrounding anatomical structures and position relationships thereof respectively by using the plurality of temporomandibular joint two-dimensional measurement films; and
fifth step: outputting a measurement and evaluation result, and generating a measurement report and a display image to provide data for clinical diagnosis and treatment.

Optionally, in the digital three-dimensional measurement and evaluation method for the temporomandibular joints, establishing the standardized measurement and evaluation procedure is characterized in that, a method for correcting the three-dimensional cone beam CT image data into the standard head position in the second step specifically includes, but is not limited to: a three-dimensional coordinate system registration method and a natural head position registration method.

Optionally, in the digital three-dimensional measurement and evaluation method for the temporomandibular joints, establishing the standardized measurement and evaluation procedure is characterized in that, after the three-dimensional cone beam CT image data are subjected to head position correction through the natural head position registration method in the second step, at a sitting position, an orbitomeatal connection line is parallel to the ground plane, and a connection line of frontozygomatic sutures on two sides is parallel to the ground plane, and at a supine position, the connection line of the frontozygomatic sutures on two sides is also parallel to the ground plane.

Optionally, a maximum axial plane of condylar processes is determined first, that is, parallel to a Frankfort horizontal plane, scanning is performed layer by layer from tops of the condylar processes to sigmoid notches, and condylar process axial layer sections with maximum inner and outer diameters of the condylar processes are selected to generate (1) a film of a maximum axial view of the condylar processes (double sides);
in a continuous section perpendicular to the maximum inner and outer diameters of the condylar processes, a section passing through a midpoint of the maximum inner and outer diameters of the condylar processes is selected as a central oblique sagittal plane of the condylar processes to generate (2) a film of a central oblique sagittal view of the condylar processes (double sides); and
in a continuous section perpendicular to a maximum axial plane of the condylar processes and the central oblique sagittal plane of the condylar processes, a section passing through the maximum inner and outer diameters of the condylar processes is selected as a central oblique coronal plane of the condylar processes to generate (3) a film of a central oblique coronal view of the condylar processes (double sides).

### 2) Establishing a Standard Measurement Method

in the above fourth step of measuring and evaluating the temporomandibular joints on both sides and the surrounding anatomical structures and position relationships thereof respectively by using the plurality of temporomandibular joint two-dimensional measurement films, mark points and reference lines on each two-dimensional measurement film, as well as a line distance between points, a distance between points and lines and an intersection angle of lines are stipulated as measurement indexes. The temporomandibular joint structures on both sides and the position relationship are evaluated by using these indexes in a sequence of determining the points first, then determining the lines and then measuring the line distance and the angle, so that the set of standard measurement method is created.

Optionally, in the digital three-dimensional measurement and evaluation method for the temporomandibular joints, establishing the standard measurement method is characterized in that, the mark points determined on the different two-dimensional measurement films specifically include, but are not limited to: (1) a film of a maximum axial view of condylar processes: a most medial point of condylar processes on an axial plane, a most lateral point of the condylar processes on the axial plane, and a geometric center of the condylar processes; (2) a film of a central oblique sagittal view of condylar processes: a condyle center, a superior condyle, a maximum point of a fossa, an anterior condyle, an anterior fossa, a posterior condyle, a posterior fossa, a minimum point of an articular tubercle, a last point of a posterior fossa, and a lowest point of a mandible sigmoid notch; and (3) a film of a central oblique coronal view of condylar processes: a medial condyle on a coronal plane, a lateral condyle on the coronal plane, a medial point of a condyle top, a middle point of the condyle top, a lateral point of the condyle top, a medial point of a fossa, a middle point of the fossa, and a lateral point of the fossa.

Optionally, in the digital three-dimensional measurement and evaluation method for the temporomandibular joints, establishing the standard measurement method is characterized in that, the reference lines determined on the different two-dimensional measurement films specifically include, but are not limited to: (1) a film of a maximum axial view of condylar processes: a mediolateral axis of condylar processes on an axial plane, and a middle sagittal line; (2) a film of a central oblique sagittal view of condylar processes: a best-fit line of an articular tubercle's posterior oblique, a top-roof line of the articular tubercle's posterior oblique, a best-fit line of a mandibular fossa's posterior oblique, a long axis of a condylar head, and a long axis of the mandible ramus on a sagittal plane; and (3) a film of a central oblique coronal view of condylar processes: a long axis of a mandible ramus on a coronal plane, and a mediolateral axis of a condyle on the coronal plane.

Optionally, in the digital three-dimensional measurement and evaluation method for the temporomandibular joints, establishing the standard measurement method is characterized in that, the measurement indexes determined on the different two-dimensional measurement films specifically include, but are not limited to: (1) a film of a maximum axial view of condylar processes: a length of condyle processes on an axial plane, a distance from a geometric center of the condylar to the MS, an anteroposterior distance between geometric centers of both condylar, a superior inferior distance between the geometric centers of both condylar, and an inclination of a mediolateral axis of the condyle; (2) a film of a central oblique sagittal view of condylar processes: a width of condylar processes, a height of condylar processes, a condylar head angle, an inclination of a mandibular ramus, an anterior joint space, a posterior joint space, a superior joint space, a length of an articular tubercle's posterior oblique, an inclination of an articular tubercle, a width of a mandibular fossa, a depth of a mandibular fossa, and a glenoid fossa divergence angle; and (3) a film of a central oblique coronal view of condylar processes: a length of condyle processes on a coronal plane, a medial joint space, a lateral joint space, a middle joint space, a horizontal inclination of a mediolateral axis of condyles, and a vertical inclination of a mandible ramus.

### 3) Standard Measurement Report and Customized Display of Image

measurement data of the temporomandibular joints on left and right sides are displayed correspondingly in the measurement report to facilitate evaluation of symmetry of joint structures on both sides as well as forms and positions; and the display image of each two-dimensional measurement film contains information of measurement data of the mark points, the reference lines, the line distance and the angle, and a display range and contents can be customized.

Optionally, in the digital three-dimensional measurement and evaluation method for the temporomandibular joints, as for the standard measurement report and customized display of the image, when a measurement result is output, a measurement report table with the measurement indexes of the joints on the left and right sides displayed correspondingly and the display image are generated, and customized display contents specifically include, but are not limited to: display of images, display of the mark points, display of the reference lines, display of the line distance index or display of the angle index, and comprehensive display, etc. The combination of graphics and text facilitates comprehensive analysis and understanding by clinical doctors.

In the solution of the present application, a digital three-dimensional measurement scheme is established on the basis of a measurement system, from collecting the cone beam CT data, to registration in three-dimensional directions, to generating two-dimensional plans of the temporomandibular joints, as well as determining and measuring the relevant points, lines, surfaces, and angles of each plan, and finally outputting measurement and evaluation results. The present application has developed a complete three-dimensional measurement and evaluation system for the temporomandibular joints, including the determination of different planes in the three-dimensional structure, as well as the determination of relevant mark points, reference lines, measurement line distances, and angles on different planes.

The present application has at least the following advantages:
1. The complete and systematic three-dimensional measurement and evaluation system for the temporomandibular joints established in the present application includes the standardized evaluation procedure. The standardized evaluation procedure includes three-dimensional cone beam CT image acquisition, head position correction of the image data through three-dimensional registration, and generation of the plurality of temporomandibular joint two-dimensional measurement films in three-dimensional directions under head position correction conditions, and measurement and evaluation of the anatomical structures and position relationships of the temporomandibular joints on two sides are completed on the two-dimensional measurement films. This set of standardized evaluation procedure ensures high repeatability and good longitudinal comparability, which facilitates horizontal comparisons of structures between different patients and between the temporomandibular joints on both sides of the same patient under the same conditions, as well as longitudinal comparisons of the same patient at different times.
2. The complete and systematic three-dimensional measurement and evaluation system for the temporomandibular joints established in the present application further includes the standard measurement method. This standard measurement method measures and comprehensively evaluates the temporomandibular joints on both sides from three dimensions: the maximum axial plane of the condylar processes, the central oblique sagittal plane of the condylar processes, and the central oblique coronal plane of the condylar processes; and this standard measurement method establishes a series of measurement mark points and measurement indexes for temporomandibular joint heads-condylar process and temporomandibular joint fossa-temporal bone joint fossa and temporomandibular joint spaces, which can truly achieve a quantitative evaluation of the temporomandibular joints and surrounding three-dimensional structures.
3. The present application establishes a set of complete and systematic three-dimensional measurement and evaluation system for the temporomandibular joints, which can provide an effective tool for collecting a large sample size of healthy temporomandibular joint image data and establishing a standard normal value range of temporomandibular joint structures, and brings new development to the evaluation of the temporomandibular joint structures and the position relationship on the basis of the three-dimensional cone beam CT image data.
4. The present application establishes the set of complete and systematic three-dimensional measurement and evaluation system for the temporomandibular joints, which changes the previous situation of manual point determination and manual measurement for the temporomandibular joints and reduces systematic errors of manual measurement; and the current situation where different researchers focus on different evaluations is changed, and a more comprehensive and powerful tool is provided for precise and in-depth scientific research. At the same time, the current situation where precise measurement of the temporomandibular joints is only applied in the scientific research field is changed, providing a more effective tool for clinical diagnosis and treatment of temporomandibular joint diseases.
5. The present application establishes the set of complete and systematic three-dimensional measurement and evaluation system for the temporomandibular joints, which changes a traditional qualitative evaluation method in clinical practice, and by quantitatively evaluating the anatomical structures of the temporomandibular joint heads, fossa and joint spaces, a more accurate imaging basis is provided for the diagnosis of the temporomandibular joint diseases, such as abrasion of the condylar processes/joint space narrowing, a specific abrasion amount, a height reduction amount, and a joint space narrowing amount, providing a scientific method for evaluating the severity of the temporomandibular joint diseases.
6. The present application establishes the complete and systematic three-dimensional measurement and evaluation system for the temporomandibular joints, which can provide a longitudinal comparative evaluation for the progression of organic changes, bone remodeling, and joint space changes in the temporomandibular joints, and provide a scientific method for long-term follow-up observation of the temporomandibular joint diseases.
7. The present application establishes the complete and systematic three-dimensional measurement and evaluation system for the temporomandibular joints, including the measurement report with measurement data of the temporomandibular joints on the left and right sides displayed symmetrically and customized display of images, and users of this method can be provided with clear and understandable display of the measurement results with the combination of graphics and text, which is conducive to better analyzing and understanding the manifestations of temporomandibular diseases, and can be applied to academic exchanges and clinical case reports.

### BRIEF DESCRIPTION OF THE DRAWINGS

With reference to the accompanying drawings and referring to the detailed description below, it will be easier to have a more complete understanding of the present application and easier to understand its accompanying advantages and features, in which:

FIG. 1 schematically illustrates a flowchart of a digital three-dimensional measurement and evaluation method for temporomandibular joints according to a preferred embodiment of the present application.

FIG. 2 schematically illustrates a sagittal direction state before head position correction.

FIG. 3 schematically illustrates a sagittal direction state after head position correction.

FIG. 4 schematically illustrates a coronal direction state before head position correction.

FIG. 5 schematically illustrates a coronal direction state after head position correction.

FIG. 6 schematically illustrates a transverse state before head position correction.

FIG. 7 schematically illustrates a transverse state after head position correction.

FIG. 8 schematically illustrates a schematic diagram of a film of a maximum axial view of condylar processes of a digital three-dimensional measurement method for temporomandibular joints according to a preferred embodiment of the present application.

FIG. 9 schematically illustrates a schematic diagram for determining a film of a central oblique coronal view of condylar processes of a digital three-dimensional measurement method for temporomandibular joints according to a preferred embodiment of the present application.

FIG. 10 schematically illustrates a schematic diagram of a film of a central oblique coronal view of condylar processes of a digital three-dimensional measurement method for temporomandibular joints according to a preferred embodiment of the present application.

FIG. 11 schematically illustrates a schematic diagram for determining a film of a central oblique sagittal view of condylar processes of a digital three-dimensional measurement method for temporomandibular joints according to a preferred embodiment of the present application.

FIG. 12 schematically illustrates a schematic diagram of a film of a central oblique sagittal view of condylar processes of a digital three-dimensional measurement method for temporomandibular joints according to a preferred embodiment of the present application.

FIGS. 13-57 schematically illustrate examples of determining mark points, determining reference lines as well as measurement items.

It should be noted that the accompanying drawings are used to illustrate the present application, not to limit it. Note that the accompanying drawings representing structures may not be drawn to scale. Moreover, in the accompanying drawings, identical or similar elements are labeled with the same or similar numbers.

### DETAILED DESCRIPTION

In order to make the content of the present application clearer and easier to understand, the content of the present application is described in detail below in conjunction with specific embodiments and accompanying drawings.

The present application provides a three-dimensional digital measurement and evaluation method for temporomandibular joints. Three-dimensional image data of the temporomandibular joints captured by cone beam CT are imported, head position correction is performed, temporomandibular joint two-dimensional measurement films are generated to complete measurement and evaluation of the temporomandibular joints and surrounding structures and positions thereof, and measurement and evaluation results are output, so that data are provided for clinical diagnosis and treatment. According to the present solution, a measurement and evaluation procedure is standard and has good reliability; a two-dimensional measurement film generation method is scientific and has good repeatability; measurement items are complete and rich, such that representativeness and longitudinal comparability are good; digital measurement achieves an accurate result; and a measurement report and image display are visual and clear.

The present application will be described specifically below.

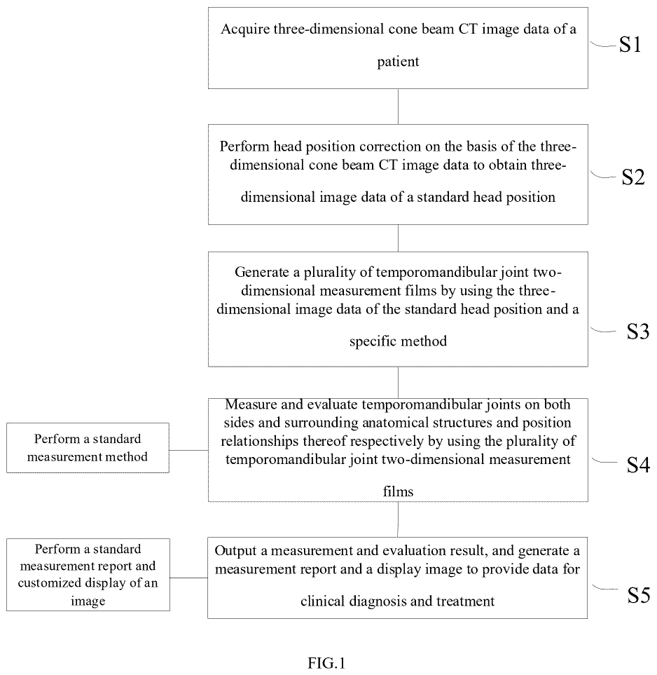

FIG. 1 schematically illustrates a flowchart of a digital three-dimensional evaluation method for temporomandibular joints according to a preferred embodiment of the present application.

As shown in FIG. 1, the digital three-dimensional evaluation method for the temporomandibular joints according to the preferred embodiment of the present application includes:

### 1) A Standardized Measurement and Evaluation Procedure is Established

first step S1: three-dimensional cone beam CT image data of a patient are obtained; and
second step S2: head position correction is performed on the basis of the three-dimensional cone beam CT image data to obtain three-dimensional image data of a standard head position.

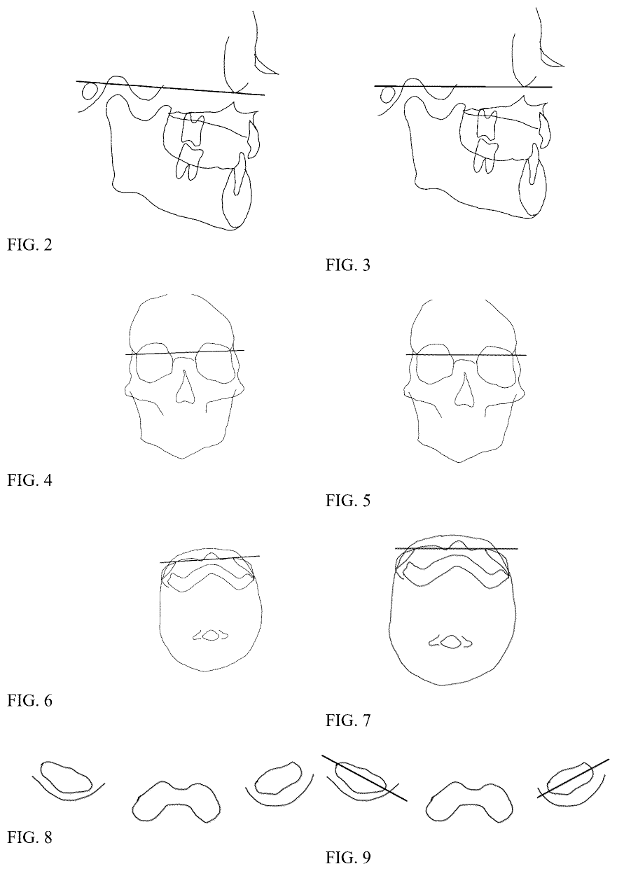

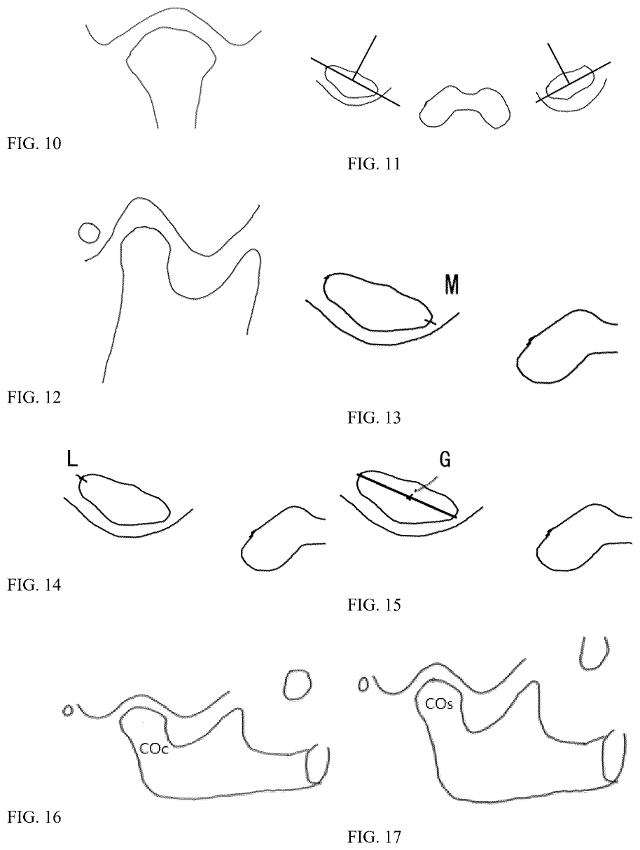

Head position correction refers to converting a natural head position during CBCT imaging into a standard head position with good repeatability. Taking a natural head position registration method as an example below, the head position is corrected (as shown in FIG. 2, the head is slightly lowered, and an orbitomeatal connection line is not parallel to the ground), and the head position is rotated to the standard head position through a linear parallel or perpendicular operation (as shown in FIG. 3, after head position correction, the orbitomeatal connection line is parallel to the ground, so the head position is changed from the original lowered head to the standard head position). Sagittal-direction rotation, coronal-direction rotation and transverse-direction rotation are performed respectively in three-dimensional directions.

Optionally, when head position correction is performed, at a sitting position, the orbitomeatal connection line is parallel to the ground plane, and a connection line of frontozygomatic sutures on two sides is parallel to the ground plane, and at a supine position, the connection line of the frontozygomatic sutures on two sides is also parallel to the ground plane. For example, a registration operation includes: two points are determined to form a connection line, such that the connection line is parallel to the ground or perpendicular to the ground, and the head position is adjusted by enabling the skull to rotate following this connection line and finally becomes direct.

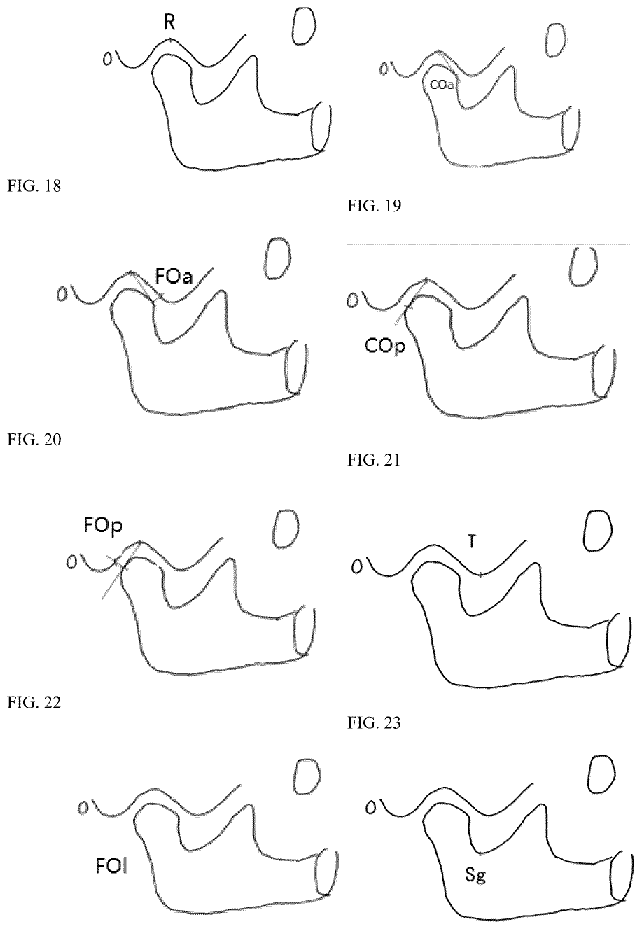

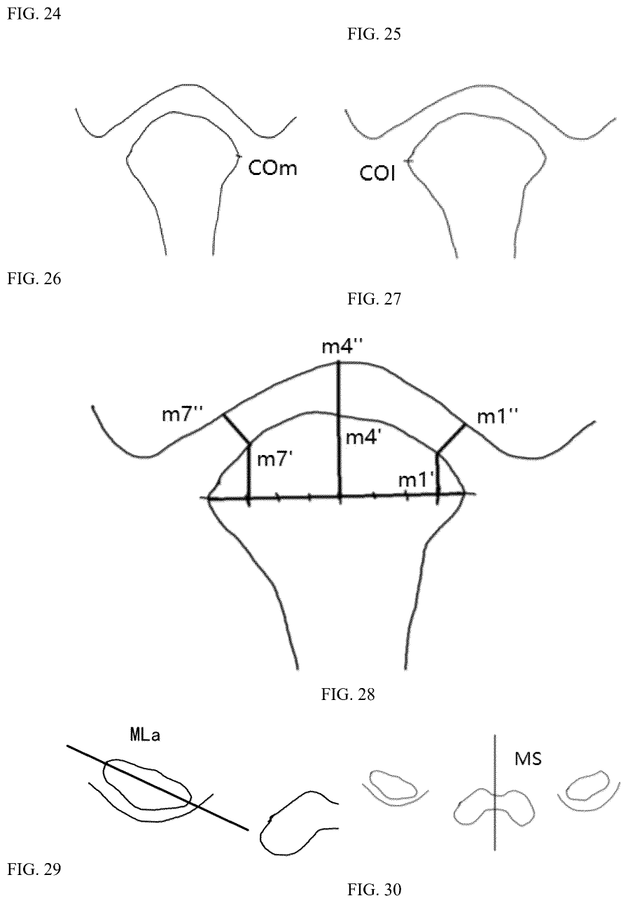

FIG. 4 schematically illustrates a coronal direction state before head position correction. At the moment, the head is slightly tilted, and the connection line of the frontozygomatic sutures on two sides is not parallel to the ground. FIG. 5 schematically illustrates a coronal forward state after head position correction. After head position correction, the connection line of the frontozygomatic sutures on two sides is parallel to the ground, so that the head position changes from the original tilted head to a direct standard head position.

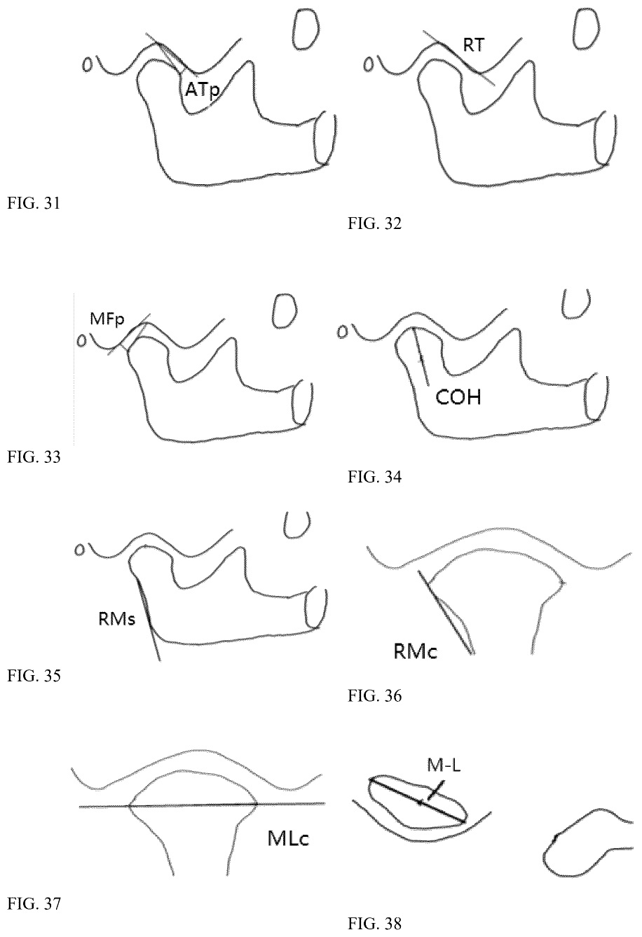

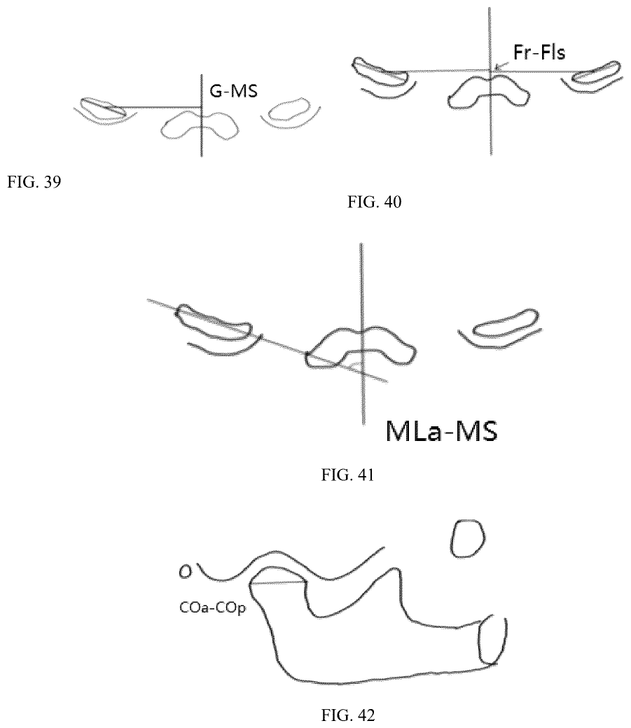

FIG. 6 schematically illustrates a transverse state before head position correction, a horizontal plane from feet to the head, or a cross section, namely the supine position. The previous head position is slightly to the right, and the connection line of the frontozygomatic sutures on two sides is not parallel to the ground. FIG. 7 schematically illustrates a transverse state before head position correction. After head position correction, at the supine position, the connection line of the frontozygomatic sutures on two sides is parallel to the ground. In this way, the head position changes from originally tilting to the right to the direct standard head position.

In this way, the head position becomes the standard head position through calibration in the three directions. This is one of the methods for head position correction.

The standard head position is obtained after head position correction. For example, originally, when a patient is taking cone beam CT, the head is slightly turned to the right, with a side higher than the other side, and the head position is re-corrected from this “natural but not direct” position to the “standard horizontal and vertical” head position through horizontal, vertical, and transverse directions. Precise films can be generated with such head position. There are other ways for head position correction, which are not exhaustively listed here.
Third step S3: the plurality of temporomandibular joint two-dimensional measurement films are generated by using the three-dimensional image data of the standard head position and a specific method.

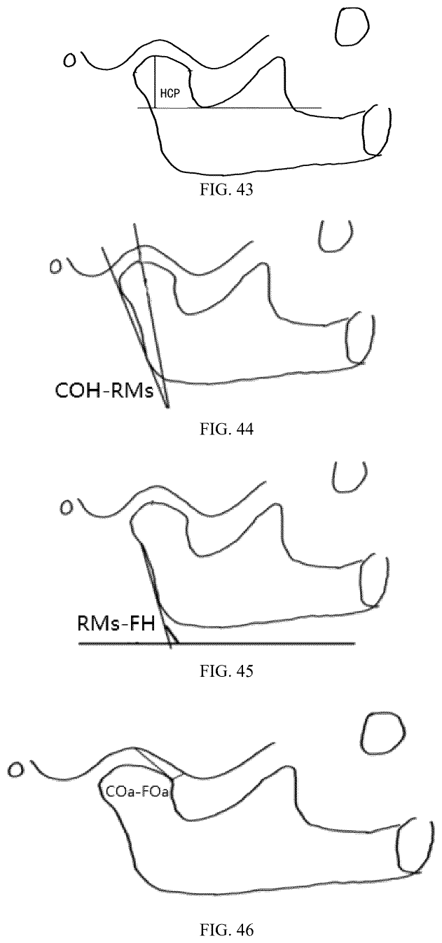

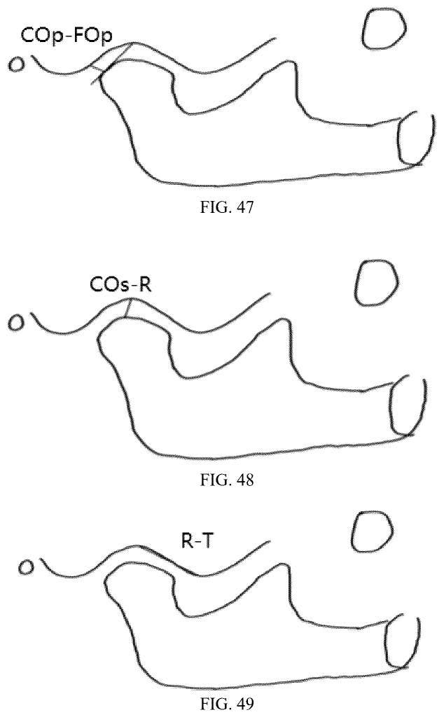

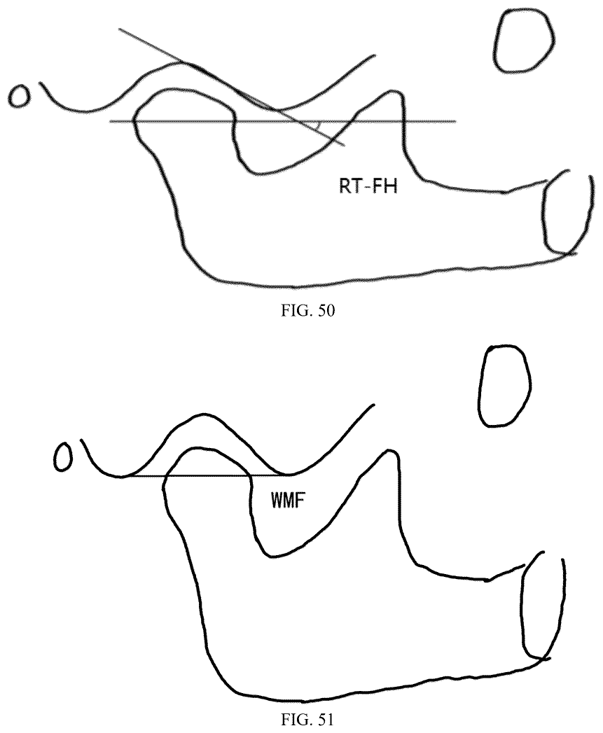

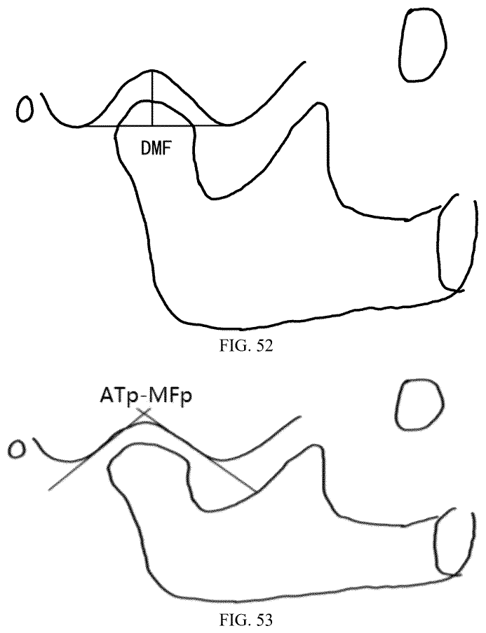

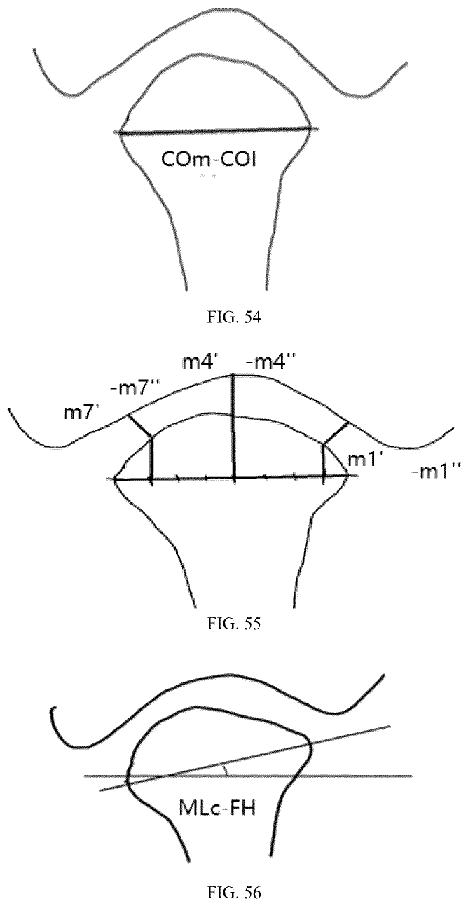

For example, the third step of generating the temporomandibular joint two-dimensional measurement films specifically includes:
first, parallel to a Frankfort horizontal plane, scanning is performed layer by layer from tops of condylar processes to sigmoid notches, and condylar process axial layer sections with maximum inner and outer diameters of the condylar processes on left and right sides are selected respectively to generate (1) a film of a maximum axial view of the condylar processes (double sides) (as shown in FIG. 8);
in a continuous section perpendicular to a maximum axial plane of the condylar processes, a section passing through the maximum inner and outer diameters of the condylar processes is selected as a central oblique coronal plane of the condylar processes (the principle for drawing is as shown in FIG. 9) to generate (2) a film of a central oblique coronal view of the condylar processes (double sides) (FIG. 10 illustrates the film of the central oblique coronal view of the condylar process on the left side or the right side); and
in a continuous section perpendicular to the maximum inner and outer diameters of the condylar processes, a section passing through a midpoint of the maximum inner and outer diameters of the condylar processes is selected as a central oblique sagittal plane of the condylar processes (the principle for drawing is as shown in FIG. 11) to generate (3) a film of a central oblique sagittal view of the condylar processes (double sides) (FIG. 12 illustrates the film of the central oblique sagittal view of the condylar process on the left side or the right side).

Therefore, the plurality of temporomandibular joint two-dimensional measurement films are generated, including, but not limited to, a film of a maximum axial view of the condylar process on the left side, a film of a central oblique sagittal view of the condylar process on the left side, a film of a central oblique coronal view of the condylar process on the left side, a film of a maximum axial view of the condylar process on the right side, a film of a central oblique sagittal view of the condylar process on the right side, and a film of a central oblique coronal view of the condylar process on the right side.
Fourth step S4: the temporomandibular joints on both sides and surrounding anatomical structures and position relationships thereof are measured and evaluated respectively by using the plurality of temporomandibular joint two-dimensional measurement films.

Optionally, in the fourth step of measuring and evaluating three-dimensional forms and positions of the temporomandibular joints, mark points are determined first, reference lines are then determined, and then various numerical values such as a line distance between points, a distance between points and lines, and an intersection angle of lines are measured.
Fifth step S5: a measurement and evaluation result is output, and a measurement report and a display image are generated to provide data for clinical diagnosis and treatment.

Optionally, in the fifth step S5, effects are displayed in a bilateral symmetry mode while the measurement and evaluation result is output. For example, the result is output according to different positions such as joint heads, fossa, joint spaces and articular tubercles respectively; or, the result is output according to different planes such as the central oblique sagittal plane of the condylar processes, the central oblique coronal plane of the condylar processes, and the maximum axial plane of the condylar processes respectively; or, the result is output according to the line distance and the angle respectively. This standardized measurement and evaluation procedure and the standard measurement method improve the accuracy and completeness of measurement and evaluation of the temporomandibular joints.

In the solution of the present application, a digital three-dimensional measurement scheme is established on the basis of the measurement system, from collecting the cone beam CT data, to head position correction, to generating the respective two-dimensional measurement films of the temporomandibular joints, as well as determining and measuring the relevant points, lines, surfaces, and angles of each plan, and finally outputting the measurement and evaluation results. The present application has developed a complete three-dimensional measurement and evaluation system for the temporomandibular joints, including the determination of different planes in the three-dimensional structure, as well as the determination of relevant mark points, reference planes, measurement line distances, and angles on different planes.

### 2) A Standard Measurement Method is Established

in the above fourth step of measuring and evaluating the temporomandibular joints on both sides and the surrounding anatomical structures and position relationships thereof respectively by using the plurality of temporomandibular joint two-dimensional measurement films, the mark points and the reference lines on each two-dimensional measurement film are stipulated, and the line distance between the points, the distance between the points and the lines and the intersection angle of the lines are stipulated as measurement indexes. The temporomandibular joint structures on both sides and the position relationships are evaluated by using these indexes in a sequence of determining the points first, then determining the lines and then measuring the line distance and the angle, so that the set of standard measurement method is created.

For example, as described above, measurement items include: the film of the maximum axial view of the condylar processes, the film of the central oblique sagittal view of the condylar processes, and the film of the central oblique coronal view of the condylar processes.

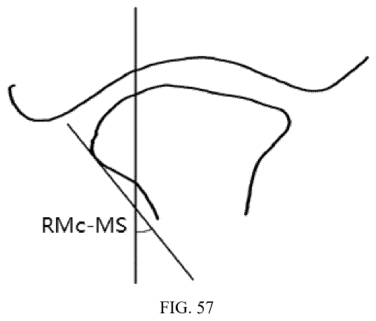

For example, point determination includes:
for instance, as for the film of the maximum axial view of the condylar processes (with a single side as an example):
(1) M: The most medial point of a condylar process on the axial plane (FIG. 13).
(2) L: The most lateral points of a condylar process on the axial plane (FIG. 14).
(3) G: Geometric center of a condylar process, a medial point of the mediolateral axis of a condylar process on the axial plane (FIG. 15).

For example, as for the film of the central oblique sagittal view of the condylar processes (with a single side as an example):
(1) COc: Condyle center, suitable for the condylar process, passing through the center of a largest circle of COs. (FIG. 16)
(2) COs: Superior condyle (as shown in FIG. 17).
(3) R: Maximum point of the fossa (as shown in FIG. 18).
(4) COa: Anterior Condyle (as shown in FIG. 19), making a tangent line to an anterior edge of the condylar process at a point R, with a point of tangency being COa.
(5) FOa: Anterior Fossa (as shown in FIG. 20), making an R-COa vertical line at a point COa, intersecting with an articular tubercle posterior oblique at a point FOa. After the point COa is determined, it is required to make the R-COa vertical line at the point COa.
(6) COp: Posterior Condyle (as shown in FIG. 21), making a tangent line to a posterior edge of the condylar process at a point R, with a point of tangency being COp.
(7) FOp: Posterior Fossa (as shown in FIG. 22), making an R-COp vertical line at the point Cop, intersecting with an articular tubercle fossa posterior oblique at a point FOp. After the point COp is determined, it is required to make the R-COp vertical line at the point COp.
(8) T: Minimum point of the articular tubercle (as shown in FIG. 23).
(9) FOI: Last point of posterior Fossa (as shown in FIG. 24), a lowest point of the fossa posterior oblique.
(10) Sg: Sigmoid notch (as shown in FIG. 25).

For example, as for the film of the central oblique coronal view of the condylar processes (with a single side as an example):
(1) COm: Medial Condyle on the coronal plane (as shown in FIG. 26).
(2) COl: Lateral Condyle on the coronal plane (as shown in FIG. 27).
(3) m1′: Intersection between perpendicular line of M-L which pass through m1 and condyle top (FIG. 28).

m1: Medial ⅛ point, ⅛ point within COm-COl, a first equal division point within eight equal division points of the long axis of the condylar head, a first equal division point within eight equal division points of a COm-COl connection line.
(4) m4′: Intersection between perpendicular line of M-L which pass through m4 and condyle top (FIG. 28).

m4: Medial ½ point, medial point of COm-COl, a fourth equal division point within eight equal division points of the long axis of the condylar head, a fourth equal division point (medial point) within eight equal division points of the COm-COl connection line.
(5) m7′: Intersection between perpendicular line of M-L which pass through m7 and condyle top (FIG. 28).

m7: Medial ⅞ point, ⅞ point within COm-COl, a seventh equal division point within eight equal division points of the long axis of the condylar head, a seventh equal division point (exterior first equal division point) within eight equal division points of the COm-COl connection line.
(6) m1″: Intersection between perpendicular line of condyle top which pass through m1′ and fossa (FIG. 28).
(7) m4″: Intersection between perpendicular line of condyle top which pass through m4′ and fossa (FIG. 28).
(8) m7″: Intersection between perpendicular line of condyle top which pass through m7′ and fossa (FIG. 28).

For example, line determination includes:
for instance, as for the film of the maximum axial view of the condylar processes
(1) MLa: The mediolateral axis of a condylar process on the axial plane (FIG. 29).
(2) MS: Middle sagittal line (FIG. 30).

For example, as for the film of the central oblique sagittal view of the condylar processes
(1) ATp: Best-fit line of the articular tubercle's posterior oblique. A tangent line passing through a point FOa and tangent to the articular tubercle's posterior oblique (FIG. 31).
(2) RT: Top-roof line of articular tubercle, a connection line between the maximum point R of the fossa and an articular tubercle bottom point T (FIG. 32).
(3) MFp: Best-fit line of the mandibular fossa's posterior oblique, a line passing through Fop and tangent to a fossa back wall (FIG. 33).
(4) COH: Long axis of the condylar head, a connection line between a highest point COs of the condylar process and a central point COs of the condylar process as the long axis of the condylar head (FIG. 34).
(5) RMs: Long axis of the mandible ramus on the sagittal plane, used as a mandible ramus long axis (as shown in FIG. 35).

For example, as for the film of the central oblique coronal view of the condylar processes
(1) RMc: Long axis of the mandible ramus on the coronal plane, used as a mandible ramus long axis (FIG. 36).
(2) MLc: Mediolateral axis of condyle on the coronal plane, a long axis passing through COm and COl (FIG. 37).

For example, the measurement items include:
for instance, as for the film of the maximum axial view of the condylar processes
(1) M-L (mm): Length of condyle process (LCP) on the axial plane, a distance between points M and L (FIG. 38)
(2) G-MS (mm): Distance from geometric center of the condylar to the MS, a vertical distance from a geometric center G to a middle sagittal line of the condylar process, a distance between points G and F (FIG. 39).

F: Foot point from G to MS, making a vertical line of a median sagittal plane at the geometric center G of the condylar process, with a foot point being F.
(3) Fr-Fls (mm): Anteroposterior distance between the geometric centers of both condylar, a horizontal distance of projections of points Fr and Fl on the sagittal plane, being positive if Fr is in front, otherwise being negative (FIG. 40).
(4) Fr-Flc (mm): Superiorinferior distance between the geometric centers of both condylar, a vertical distance of projections of points Fr and Fl on the coronal plane, being positive if Fr is above, otherwise being negative
(5) MLa-MS (°): Inclination of mediolateral axis of condyle, an included angle (inner anterior angle) between the long axis of the condylar head and the median sagittal line (FIG. 41).

For example, as for the film of the central oblique sagittal view of the condylar processes
(1) COa-COp (mm): Width of condylar process, a distance between points COa and Cop (FIG. 42).
(2) HCP (mm): Height of condylar process, a vertical distance between a superior condyle COs and the Sigmoid notch Sg (FIG. 43).
(3) COH-RMs (°): Condylar head angle, angle between condylar head and mandibular ramus, an included angle (upper and lower angles) of the long axis of the condylar head COH and a mandibular ramus posterior tangent line RMs (FIG. 44).
(4) RMs-FH (°): Inclination of the mandibular ramus, an included angle (anterior upper included angle) of the mandibular ramus posterior tangent line RMs and a horizontal line FH (as shown in FIG. 45).
(5) COa-FOa (mm): Anterior joint space (Ajs), a distance between points COa and FOa (FIG. 46).
(6) COp-FOp (mm): Posterior joint space (Pjs), a distance between points COp and FOp (FIG. 47).
(7) COs-R (mm): Superior joint space (Sjs), a distance between points COs and R (FIG. 48).
(8) R-T (mm): Length of the articular tubercle's posterior oblique, a distance between points R-T (FIG. 49).
(9) RT-FH (°): Inclination of articular tubercle (IRT) (top-bottom method), an anterior lower intersection angle of a line RT and the horizontal line FH (FIG. 50).
(10) WMF (mm): Width of the mandibular fossa, a distance between points FOl and T (FIG. 51).
(11) DMF (mm): Depth of the mandibular fossa, equal to the articular tubercle's height, a vertical distance between an uppermost point R of the fossa and FOl-T (FIG. 52).
(12) ATp-MFp (°): Glenoid fossa divergence angle. An included angle between the best-fit line of the articular tubercle's anterior oblique ATp and the best-fit line of the mandibular fossa's posterior oblique MFp (FIG. 53).

For example, as for the film of the central oblique coronal view of the condylar processes
(1) COm-COl (mm): Length of condyle process (LCP) on the coronal plane, a distance between points COm and COl (FIG. 54).
(2) m1′-m1″ (mm): Medial joint space (Mjs), a distance between points m1′ and m1″ (FIG. 55).
(3) m7′-m7″ (mm): Lateral joint space (Ljs), a distance between points m7′ and m7″, and if m7″ is unavailable (the point COl of the condylar process is located outside the fossa), replaced with a shortest distance from an outermost lower point of the fossa to the condylar process (FIG. 55).
(4) m4′-m4″ (mm): Middle joint space (Mis), a distance between points m4′ and m4″ (FIG. 55)
(5) MLc-FH (°): Horizontal inclination of mediolateral axis of condyle, a horizontal inclination of the inner and outer diameters of the condylar process, an included angle between the mediolateral axis of the condyle MLc and the horizontal line FH (an included angle of inward and outward directions, being positive if COm is above COl, otherwise being negative) (FIG. 56).
(6) RMc-MS (°): Vertical inclination of mandible ramus, an included angle between the long axis of the mandible ramus RMc and the median sagittal line MS (an included angle of upward and downward directions, being positive if the long axis of the mandible ramus is inward, otherwise being negative) (FIG. 57).

Of course, point determination, line determination, the measurement items, etc. may further be performed on other measurement planes.

### 3) Standard Measurement Report and Customized Display of Image

measurement data of the temporomandibular joints on left and right sides are displayed correspondingly in the measurement report to facilitate an evaluation of symmetry of joint structures on both sides as well as forms and positions; and the display image of each two-dimensional measurement film contains information of measurement data of the mark points, the reference lines, the line distance and the angle, and a display range and contents can be customized.

The present application has at least the following advantages:
1. The complete and systematic three-dimensional measurement and evaluation system for the temporomandibular joints established in the present application includes the standardized evaluation procedure. The standardized evaluation procedure includes three-dimensional cone beam CT image acquisition, head position correction of the image data through three-dimensional registration, and generation of the plurality of temporomandibular joint two-dimensional measurement films in three-dimensional directions under head position correction conditions, and measurement and evaluation of the anatomical structure and position relationship of the temporomandibular joints on two sides are completed on the two-dimensional measurement films. This standardized evaluation procedure ensures high repeatability and good longitudinal comparability, which facilitates horizontal comparisons of structures between different patients and between the temporomandibular joints on both sides of the same patient under the same conditions, as well as longitudinal comparisons of the same patient at different times.
2. The complete and systematic three-dimensional measurement and evaluation system for the temporomandibular joints established in the present application further includes the standard measurement method. This standard measurement method measures and comprehensively evaluates the temporomandibular joints on both sides from three dimensions: the maximum axial plane of the condylar processes, the central oblique sagittal plane of the condylar processes, and the central oblique coronal plane of the condylar processes; and this standard measurement method establishes a series of measurement mark points and measurement indexes for temporomandibular joint heads-condylar process and temporomandibular joint fossa-temporal bone joint fossa and temporomandibular joint spaces, which can truly achieve quantitative evaluation of the temporomandibular joints and surrounding three-dimensional structures.
3. The present application establishes a set of complete and systematic three-dimensional measurement and evaluation system for the temporomandibular joints, which can provide an effective tool for collecting a large sample size of healthy temporomandibular joint image data and establishing a standard normal value of temporomandibular joint structures, and brings new development to the evaluation of the temporomandibular joint structures and the position relationship on the basis of the three-dimensional cone beam CT image data.
4. The present application establishes the set of complete and systematic three-dimensional measurement and evaluation system for the temporomandibular joints, which changes the previous situation of manual point determination and manual measurement for the temporomandibular joints and reduces systematic errors of manual measurement; and the current situation where different researchers focus on different evaluations is changed, and a more powerful tool is provided for precise and in-depth scientific research. At the same time, the current situation where precise measurement of the temporomandibular joints is only applied in the scientific research field is changed, providing a more effective tool for clinical diagnosis and treatment of temporomandibular joint diseases.
5. The present application establishes the set of complete and systematic three-dimensional measurement and evaluation system for the temporomandibular joints, which changes a traditional qualitative evaluation method in clinical practice, and by quantitatively evaluating the anatomical structures of the temporomandibular joint heads, fossa and joint spaces, a more accurate imaging basis is provided for the diagnosis of the temporomandibular joint diseases, such as abrasion of the condylar processes/joint space narrowing, a specific abrasion amount, a height reduction amount, and a joint space narrowing amount, providing a scientific method for evaluating the severity of the temporomandibular joint diseases.
6. The present application establishes the complete and systematic three-dimensional measurement and evaluation system for the temporomandibular joints, which can provide a longitudinal comparative evaluation for the progression of organic changes, bone remodeling, and joint space changes in the temporomandibular joints, and provide a scientific method for long-term follow-up observation of the temporomandibular joint diseases.
7. The present application establishes the complete and systematic three-dimensional measurement and evaluation system for the temporomandibular joints, including the measurement report with measurement data of the temporomandibular joints on the left and right sides displayed symmetrically and customized display of images, and users of this method can be provided with clear and understandable display of the measurement results with the combination of graphics and text, which is conducive to better analyzing and understanding the manifestations of temporomandibular diseases, and can be applied to academic exchanges and clinical case reports.

In a word, the three-dimensional measurement and evaluation method in this solution is established scientifically and accurately, such that the measurement result is precise, and the measurement solution is high in repeatability. This solution is based on head position correction performed before each measurement. In this solution, the method for generating the respective two-dimensional measurement films of three-dimensional structures of the temporomandibular joints is scientific and accurate, and imaging pictures may be stored to facilitate repeated measurement. The measurement method for the temporomandibular joints in this solution is systematic, which is embodied in that: (1) measurement contents are complete and rich, including sagittal-direction, coronal-direction and axial-direction measurement and evaluation for the temporomandibular joints, as well as measurement and evaluation for joint heads, fossa, articular tubercles and joint spaces; (2) the measurement method is scientific, the mark points (mainly anatomic mark points with good recognizability) are determined first, then the reference lines are determined, various numerical values such as the line distance between the points, the distance between the points and the lines, and the intersection angle of lines are measured, and this standardized measurement procedure and the standard measurement method improve the accuracy and completeness of three-dimensional measurement of the temporomandibular joints; and (3) the measurement result in this solution is output in a visible display mode and is clear, and although there are various measurement items, the use of symmetrical, grouped, and clear output manners facilitates clinical comparative analysis of structures and positions of the temporomandibular joints on both sides.

In addition, it should be noted that, unless otherwise specified, the terms “first”, “second”, “third” and other descriptions in the specification are only used to distinguish various components, elements, steps, etc. in the specification, and are not used to represent logical or sequential relationships, etc. between the various components, elements and steps.

It will be appreciated that, although the present application has been disclosed as above through preferred embodiments, the above embodiments are not intended to limit the present application. For any person skilled in the art, without departing from the scope of the technical solution of the present application, the technical content disclosed above may be used to make many possible changes and modifications to the technical solution of the present application, or the technical solution may be modified into equivalent embodiments with equivalent changes. Therefore, any simple amendments, equivalent changes and modifications made to the above embodiments in accordance with the technical essence of the present application that do not depart from the content of the technical solution of the present application still fall within the scope of protection of the technical solution of the present application.

1. A digital three-dimensional measurement and evaluation method for temporomandibular joints, comprising:
performing a standardized measurement and evaluation procedure:
first step: acquiring three-dimensional cone beam CT image data of a patient;
second step: performing head position correction on the basis of the three-dimensional cone beam CT image data to obtain three-dimensional image data of a standard head position;
third step: generating a plurality of temporomandibular joint two-dimensional measurement films by using the three-dimensional image data of the standard head position and a specific method;
fourth step: measuring and evaluating temporomandibular joints on both sides and surrounding anatomical structures and position relationships thereof respectively by using the plurality of temporomandibular joint two-dimensional measurement films; and
fifth step: outputting a measurement and evaluation result, and generating a measurement report and a display image to provide data for clinical diagnosis and treatment.

2. The digital three-dimensional measurement and evaluation method for the temporomandibular joints according to claim 1, further comprising:
performing a standard measurement method, comprising:
in the fourth step of measuring and evaluating the temporomandibular joints on both sides and the surrounding anatomical structures and position relationships thereof respectively by using the plurality of temporomandibular joint two-dimensional measurement films, stipulating mark points and reference lines on each two-dimensional measurement film, and stipulating a line distance between points, a distance between points and lines and an intersection angle of lines as measurement indexes; and evaluating the temporomandibular joint structures on both sides and the position relationship by using the comprehensive and rich measurement indexes in a sequence of determining the points first, then determining the lines and then measuring the line distance and the angle.

3. The digital three-dimensional measurement and evaluation method for the temporomandibular joints according to claim 1, further comprising:
performing a standard measurement report and customized display of the image, comprising:
displaying measurement data of the temporomandibular joints on left and right sides correspondingly in the measurement report to facilitate evaluation of symmetry of joint structures on both sides as well as forms and positions, wherein the display image of each two-dimensional measurement film contains information of measurement data of the mark points, the reference lines, the line distance and the angle, and a display range and contents are able to be customized.

4. The digital three-dimensional measurement and evaluation method for the temporomandibular joints according to claim 1, wherein, in establishing the standardized measurement and evaluation procedure, a method for correcting the three-dimensional cone beam CT image data into the standard head position in the second step specifically comprises, but is not limited to: a three-dimensional coordinate system registration method and a natural head position registration method.

5. The digital three-dimensional measurement and evaluation method for the temporomandibular joints according to claim 1, wherein, in establishing the standardized measurement and evaluation procedure, the plurality of two-dimensional measurement films generated by using the three-dimensional cone beam CT image data of the standard head position and the specific method in the third step specifically comprise, but are not limited to: a film of a maximum axial view of left and right condylar processes, a film of a central oblique sagittal view of left and right condylar processes, and a film of a central oblique coronal view of left and right condylar processes.

6. The digital three-dimensional measurement and evaluation method for the temporomandibular joints according to claim 1, wherein, in establishing the standardized measurement and evaluation procedure, the specific method for generating the two-dimensional measurement films by using the three-dimensional cone beam CT image data of the standard head position in the third step comprises:
first, parallel to a Frankfort horizontal plane, scanning layer by layer from tops of condylar processes to sigmoid notches, and selecting condylar process axial layer sections with maximum inner and outer diameters of the condylar processes on both sides respectively to generate a film of a maximum axial view of left and right condylar processes;
in a continuous section perpendicular to the maximum inner and outer diameters of the condylar processes, selecting a section passing through a midpoint of the maximum inner and outer diameters of the condylar processes as a central oblique sagittal plane of the condylar processes to generate a film of a central oblique sagittal view of left and right condylar processes; and
in a continuous section perpendicular to a maximum axial plane of the condylar processes and the central oblique sagittal plane of the condylar processes, selecting a section passing through the maximum inner and outer diameters of the condylar processes as a central oblique coronal plane of the condylar processes to generate a film of a central oblique coronal view of left and right condylar processes.

7. The digital three-dimensional measurement and evaluation method for the temporomandibular joints according to claim 1, wherein, in establishing the standard measurement method, the mark points determined on the different two-dimensional measurement films specifically comprise, but are not limited to: (1) a film of a maximum axial view of condylar processes: a most medial point of condylar processes on an axial plane, a most lateral point of the condylar processes on the axial plane, and a geometric center of the condylar processes; (2) a film of a central oblique sagittal view of condylar processes: a condyle center, a superior condyle, a maximum point of a fossa, an anterior condyle, an anterior fossa, a posterior condyle, a posterior fossa, a minimum point of an articular tubercle, a last point of a posterior fossa, and a lowest point of a mandible sigmoid notch; and (3) a film of a central oblique coronal view of condylar processes: a medial condyle on a coronal plane, a lateral condyle on the coronal plane, a medial point of a condyle top, a middle point of the condyle top, a lateral point of the condyle top, a medial point of a fossa, a middle point of the fossa, and a lateral point of the fossa.

8. The digital three-dimensional measurement and evaluation method for the temporomandibular joints according to claim 1, wherein, in establishing the standard measurement method, the reference lines determined on the different two-dimensional measurement films specifically comprise, but are not limited to: (1) a film of a maximum axial view of condylar processes: a mediolateral axis of condylar processes on an axial plane, and a middle sagittal line; (2) a film of a central oblique sagittal view of condylar processes: a best-fit line of an articular tubercle's posterior oblique, a top-roof line of the articular tubercle's posterior oblique, a best-fit line of a mandibular fossa's posterior oblique, a long axis of a condylar head, and a long axis of the mandible ramus on a sagittal plane; and (3) a film of a central oblique coronal view of condylar processes: a long axis of a mandible ramus on a coronal plane, and a mediolateral axis of a condyle on the coronal plane.

9. The digital three-dimensional measurement and evaluation method for the temporomandibular joints according to claim 1, wherein, in establishing the standard measurement method, the measurement indexes determined on the different two-dimensional measurement films specifically comprise, but are not limited to: (1) a film of a maximum axial view of condylar processes: a length of condyle processes on an axial plane, a distance from a geometric center of the condylar to the MS, an anteroposterior distance between geometric centers of both condylar, a superior inferior distance between the geometric centers of both condylar, and an inclination of a mediolateral axis of the condyle; (2) a film of a central oblique sagittal view of condylar processes: a width of condylar processes, a height of condylar processes, a condylar head angle, an inclination of a mandibular ramus, an anterior joint space, a posterior joint space, a superior joint space, a length of an articular tubercle's posterior oblique, an inclination of an articular tubercle, a width of a mandibular fossa, a depth of a mandibular fossa, and a glenoid fossa divergence angle; and (3) a film of a central oblique coronal view of condylar processes: a length of condyle processes on a coronal plane, a medial joint space, a lateral joint space, a middle joint space, a horizontal inclination of a mediolateral axis of condyles, and a vertical inclination of a mandible ramus.

10. The digital three-dimensional measurement and evaluation method for the temporomandibular joints according to claim 3, wherein, in the standard measurement report and customized display of the image, when a measurement result is output, a measurement report table with the measurement indexes of the temporomandibular joints on the left and right sides displayed correspondingly and the display image are generated, and customized display contents specifically comprise, but are not limited to: display of images, display of the mark points, display of the reference lines, display of the line distance index or display of the angle index, and combined or comprehensive display.

11. The digital three-dimensional measurement and evaluation method for the temporomandibular joints according to claim 2, further comprising:
performing a standard measurement report and customized display of the image, comprising:
displaying measurement data of the temporomandibular joints on left and right sides correspondingly in the measurement report to facilitate evaluation of symmetry of joint structures on both sides as well as forms and positions,
wherein the display image of each two-dimensional measurement film contains information of measurement data of the mark points, the reference lines, the line distance and the angle, and a display range and contents are able to be customized.

12. The digital three-dimensional measurement and evaluation method for the temporomandibular joints according to claim 2, wherein, in establishing the standard measurement method, the mark points determined on the different two-dimensional measurement films specifically comprise, but are not limited to: (1) a film of a maximum axial view of condylar processes: a most medial point of condylar processes on an axial plane, a most lateral point of the condylar processes on the axial plane, and a geometric center of the condylar processes; (2) a film of a central oblique sagittal view of condylar processes: a condyle center, a superior condyle, a maximum point of a fossa, an anterior condyle, an anterior fossa, a posterior condyle, a posterior fossa, a minimum point of an articular tubercle, a last point of a posterior fossa, and a lowest point of a mandible sigmoid notch; and (3) a film of a central oblique coronal view of condylar processes: a medial condyle on a coronal plane, a lateral condyle on the coronal plane, a medial point of a condyle top, a middle point of the condyle top, a lateral point of the condyle top, a medial point of a fossa, a middle point of the fossa, and a lateral point of the fossa.

13. The digital three-dimensional measurement and evaluation method for the temporomandibular joints according to claim 2, wherein, in establishing the standard measurement method, the reference lines determined on the different two-dimensional measurement films specifically comprise, but are not limited to: (1) a film of a maximum axial view of condylar processes: a mediolateral axis of condylar processes on an axial plane, and a middle sagittal line; (2) a film of a central oblique sagittal view of condylar processes: a best-fit line of an articular tubercle's posterior oblique, a top-roof line of the articular tubercle's posterior oblique, a best-fit line of a mandibular fossa's posterior oblique, a long axis of a condylar head, and a long axis of the mandible ramus on a sagittal plane; and (3) a film of a central oblique coronal view of condylar processes: a long axis of a mandible ramus on a coronal plane, and a mediolateral axis of a condyle on the coronal plane.

14. The digital three-dimensional measurement and evaluation method for the temporomandibular joints according to claim 2, wherein, in establishing the standard measurement method, the measurement indexes determined on the different two-dimensional measurement films specifically comprise, but are not limited to: (1) a film of a maximum axial view of condylar processes: a length of condyle processes on an axial plane, a distance from a geometric center of the condylar to the MS, an anteroposterior distance between geometric centers of both condylar, a superior inferior distance between the geometric centers of both condylar, and an inclination of a mediolateral axis of the condyle; (2) a film of a central oblique sagittal view of condylar processes: a width of condylar processes, a height of condylar processes, a condylar head angle, an inclination of a mandibular ramus, an anterior joint space, a posterior joint space, a superior joint space, a length of an articular tubercle's posterior oblique, an inclination of an articular tubercle, a width of a mandibular fossa, a depth of a mandibular fossa, and a glenoid fossa divergence angle; and (3) a film of a central oblique coronal view of condylar processes: a length of condyle processes on a coronal plane, a medial joint space, a lateral joint space, a middle joint space, a horizontal inclination of a mediolateral axis of condyles, and a vertical inclination of a mandible ramus.
